(19) United States
(12) Plant Patent Application Publication
(10) Pub. No.: US 2018/0235125 P1
(43) Pub. Date: Aug. 16, 2018

(54) Strawberry plant named 'Magellan'

(50) ;

(71) Applicant: Sweet Darling Sales, Inc., Aptos, CA (US)

(72) Inventor: John LARSE, Watsonville, CA (US)

(21) Appl. No.: 15/731,552

(22) Filed: Jun. 28, 2017

Related U.S. Application Data
(60) Provisional application No. 62/355,575, filed on Jun. 28, 2016.

(51) Int. Cl. A01H 6/74

(52) U.S. Cl. PLT/209

(57) ABSTRACT

The present invention provides a new and distinct strawberry plant designated as ‘Magellan’ (a.k.a. ‘108789’).

Latin name of the genus and species: Fragaria x ananassa.

Varietal denomination: ‘Magellan’ (a.k.a. ‘108789’).

### BACKGROUND OF THE INVENTION

The present invention relates to a new and distinct strawberry plant designated as ‘Magellan’ (a.k.a. ‘108789’). ‘Magellan’ is a short-day plant.

‘Magellan’ (a.k.a. ‘108789’) is the result of a controlled-cross between a female parent cultivar designated ‘107705’ (unpatented, proprietary cultivar) and a male parent cultivar designated ‘106861’ (unpatented, proprietary cultivar) made by the Inventor and was first fruited in Watsonville, Calif. growing fields. Following selection and during testing, the plant was originally designated ‘108789’ and subsequently named ‘Magellan’.

This new strawberry plant was asexually reproduced via runners (stolons) by the inventor at Watsonville, Calif. Asexual propagules from the original source have been tested in Watsonville growing fields and to a limited extent, grower fields in high elevation. The properties of this new strawberry plant were found to be transmissible by such asexual reproduction. This new strawberry plant is stable and reproduce true to type in successive generations of asexual reproduction.

### BRIEF SUMMARY OF THE INVENTION

This invention relates to new and distinctive strawberry plant designated as ‘Magellan’. This strawberry plant is primarily adapted to the climate and growing conditions of the central coast of California. This region provides the necessary temperatures required for it to produce a strong vigorous plant and to remain in fruit production from March through October. The nearby Pacific Ocean provides the needed humidity and moderate day temperatures and evening chilling to maintain fruit quality for the production months.

The following traits and photographs in combination distinguish strawberry plant ‘Magellan’ from known strawberry varieties. In addition, this new strawberry plant was confirmed to be a unique strawberry germplasm when tested against the California Seed & Plant Lab, Inc. (Elverta, Calif.) database using Short Sequence Repeats (SSRs). Plants for the botanical measurements in the present application were grown as annuals. Any color references are made to The Royal Horticultural Society Colour Chart, 1995 Edition, except where general terms of ordinary dictionary significance are used. The fruit produced by each new cultivar is attractive and of excellent quality.

TABLE 1
Magellen

| CharType | Characteristic | Magellan |
|---|---|---|
| General | Plant Habit | annual |
| | Plant Growth Habit | upright |
| | Plant Height | 24 cm |
| | Plant Width | 29 cm |
| | Plant Width-Crown | 3 cm |
| | Density of foliage, vigor | medium |
| | Plant vigor | moderate to high |
| Leaf | Terminal leaflet width (mm) | 79 |
| | Terminal leaflet length (mm) | 82 |
| | No. teeth/terminal leaflet: | 19 |
| | Shape of the terminal leaflet base | rounded |
| | Shape of terminal leaflet in cross- | concave |
| | section | |
| | Margin description of the terminal | serrate to crenate |
| | Color of upper side of leaves | 137A |
| | Color of lower side of leaves | 139C |
| | Leaf blistering | weak |
| | Leaf glossiness | weak |
| Limbs | Petiole length (cm) | 13 |
| | Petiole diameter (mm) | 3.89 |
| | Petiole color | 145B |
| | Petiolule length (mm) | 6 |
| | Petiolule diameter (mm) | 3.89 |
| | Attitude of hairs on | upwards |
| | petiole and pedicel | |
| | Stipule pubescence | medium to heavy |
| | Stipule length (cm) | 5 |
| | Stipule size | large |
| | Stipule width (cm) | 1.2 |
| | Stipule anthocyanin | present |
| | Stipule color (color code) | 145A |
| | Pedicel color (color code) | 145B |
| | Peduncle length (cm) | 27 |
| | Peduncle size | medium to large |
| | Peduncle attitude | erect |
| | Peduncle pubescence, attitude of hairs | Medium, upwards |
| Inflorescence | Inflorescence position relative to | above |
| | foliage | |
| | Flower arrangement of petals | touching |
| | Petal length (cm) | 1.5 |
| | Petal width (cm) | 1.5 |
| | Petal number per flower | 6 |
| | Upper Petal color | 155C |
| | Lower Petal color | 157B |
| | Calyx diameter (cm) | 3.9 |
| | Corolla diameter (cm) | 3.5 |
| | Sepal length (cm) | 1.6 |
| | Sepal width (cm) | 0.4 |
| | Time of flowering | March |
| | (50% | of plants in bloom) |
| | Shape of stigma | capitate |
| | Color of stigma | 12A |
| | Length of style (mm) | 2 |
| | Color of style | 4A |
| | Color of the ovary | 145B |
| | Length of the stamens (mm) | 5 |
| | Number of stamen | 25 |
| | Anther color | 12A |
| | Shape of anther | dorsifixed |
| | Size of anther | medium |
| | Amount of pollen | moderate |
| | Color of pollen | 4A |
| | Color of filament | 2C |
| | Length of filament (mm) | 4 |
| | Number of flowers per truss | 2 to 7 |
| Stolon | Stolon number | 3 |
| | Stolon anthocyanin | 166A |
| | Widest diameter of stolon | 4.41 |
| | At leaf attachment (mm) | |
| | Stolon color | 145B |
| Fruit | Number of fruit per truss | 2 to 5 |
| | Fruit length (cm) | 4.2 |
| | Fruit width (cm) | 4.1 |
| | Fruit skin color | 53A to 45A |
| | Fruit flesh color excluding core | 44A |
| | Fruit core length (cm) | 3.4 |
| | Fruit core width (cm) | 1.8 |
| | Fruit core color | 41A |
| | Fruit weight (g) | 26.8 |
| | Predominant fruit shape | short wedge to conic |
| | Shape difference between primary | Similar shape |
| | & secondary fruits | |
| | Width of band without of achenes | medium |
| | Fruit glossiness | medium to strong |
| | Position of achenes | |
| | Achene color | 145B |
| | Achenes per fruit | 438 |
| | Achene weight (g) | 0.26 |
| | Position of calyx | even to inserted |
| | level of adherence of calyx | strong |
| | Color of calyx | 137A |
| | Firmness of flesh | firm |
| | Evenness of flesh color | nearly even |
| | Sweetness (brix) | 9 |
| | pH | 3.27 |
| | Yield (g per plant per season) | 2675 |

### DESCRIPTION OF THE DRAWINGS

The accompanying color photographs depict various characteristics of the cultivars as nearly true as possible to make color reproductions.

FIG. 1 shows fruits of ‘Magellan’.

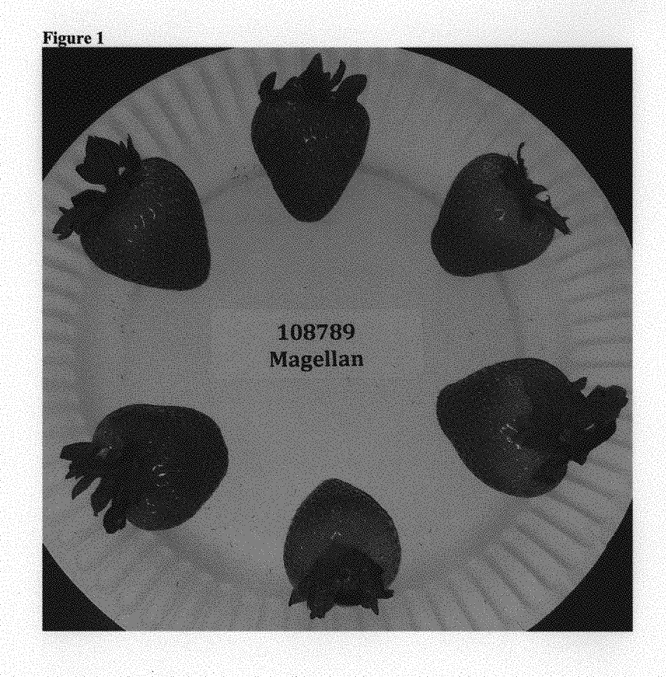

FIG. 2 shows sliced fruits of ‘Magellan’.

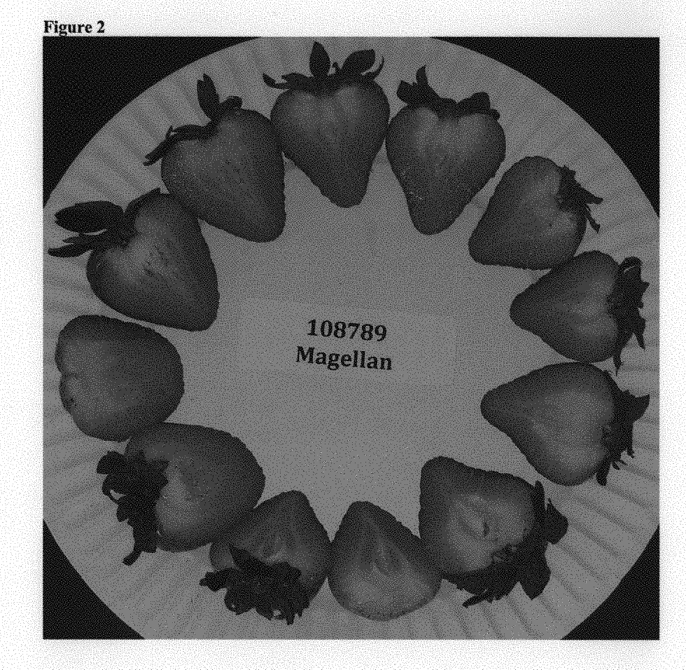

FIG. 3 shows ‘Magellan’ plants.

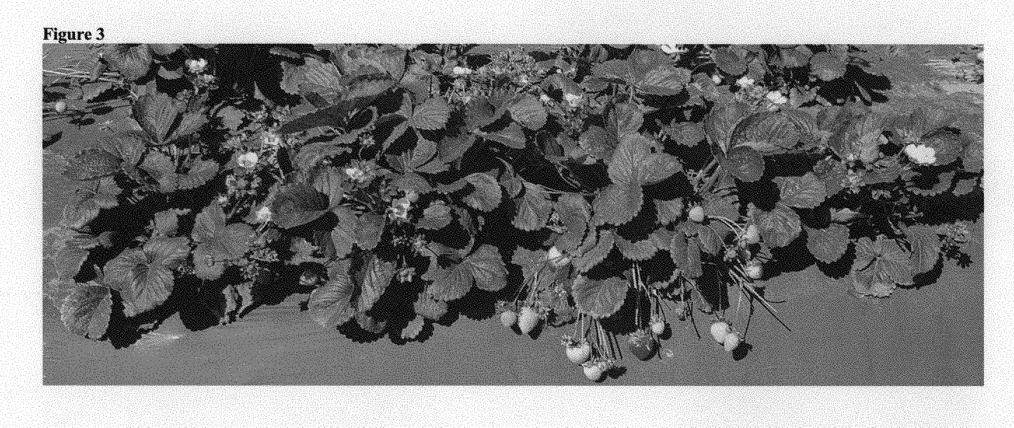

1. A new and distinct cultivar of strawberry plant named ‘Magellan’ substantially as shown and described herein.
